(19) United States
(12) Patent Application Publication
(10) Pub. No.: US 2012/0109328 A1
(43) Pub. Date: May 3, 2012

(54) Acetabular Labrum Implant

(75) Inventor: Jason D. Meridew, Warsaw, IN (US)

(73) Assignee: Biomet Manufacturing Corp., Warsaw, IN (US)

(21) Appl. No.: 12/915,366

(22) Filed: Oct. 29, 2010

(51) Int. Cl. A61F 2/32

(52) U.S. Cl. 623/22.11

(57) ABSTRACT

A labrum implant can include a body portion and an attachment member. The body portion can be formed of a biocompatible flexible polymer and can include a base, an opposed upper end opposite the base, an interior femoral head engaging side, and an opposed exterior side. The interior side can include an arcuate shape in cross-section adapted to conform to an articular surface of a femoral head. The body portion can include an arcuate shape in plan view adapted to conform to at least a portion of a rim of an acetabulum and a plurality of bores extending therethrough from the upper surface or the exterior side to the base. The attachment member can include at least a portion coupled to at least one of the bores and can be adapted to secure the implant to the acetabular rim such that the base engages the acetabular rim.

### FIELD

The present teachings relate generally to a labrum implant, and more particularly, to an acetabular labrum implant for a hip joint and associated method.

### BACKGROUND

The statements in this section merely provide background information related to the present disclosure and may not constitute prior art.

In the human anatomy, various portions of the body are interconnected through soft tissues. For example, ligaments and cartilage interconnect various portions of the anatomy to provide selected articulations of adjacent bone portions in an articulating joint. Over time, the cartilage may become weakened or damaged due to various reasons including injury, fatigue, age, disease, etc. In certain circumstances, damage to the cartilage, such as tears in the labrum in a hip joint, can be minor such that the labral tears can be reattached. In other circumstances, the labral tears can be extensive such that the damaged labrum is not capable of being repaired, which can result in a future need to replace the natural hip joint with a prosthetic hip joint.

While such a procedure to replace the natural hip joint with a prosthetic joint works for its intended purpose, there nevertheless exists a need in the art for an improved technique where the natural labrum can be replaced with a labrum implant.

### SUMMARY

This section provides a general summary of the disclosure, and is not a comprehensive disclosure of its full scope or all of its features.

In one aspect a labrum implant configured to replace at least a portion of the natural labrum about the acetabulum is provided. The labrum implant can include a body portion and an attachment member. The body portion can be formed of at least a biocompatible flexible polymer and can include a base, an opposed upper end opposite the base, an interior femoral head engaging side, and an opposed exterior side. The interior side can include an arcuate shape in cross-section adapted to conform to an articular surface of the femoral head. The body portion can include an arcuate shape in plan view adapted to conform to at least a portion of the rim of the acetabulum, and a plurality of bores extending therethrough from the upper surface or the exterior side to the base. The attachment member can include at least a portion coupled to at least one of the bores and can be adapted to secure the implant to the acetabulum such that the base engages the acetabular rim.

In another aspect a labrum implant configured to replace at least a portion of the natural labrum about the acetabulum is provided and can include a body portion and a support portion. The body portion can be formed of at least a biocompatible flexible polymer and can include a base, an opposed upper end opposite the base, an interior femoral head engaging side, and an opposed exterior side. The interior side can include a shape in cross-section adapted to conform to an articular surface of the femoral head. The body portion can include an arcuate shape in plan view adapted to conform to a rim of an acetabulum. The support portion can be formed of at least a semi-rigid biocompatible material and can include a first side adapted to engage the acetabular rim and an opposing second side fixed to the body portion. The support portion can also include an arcuate shape complimentary to the arcuate shape of the body portion. At least the first side of the support portion can include a roughened or porous surface configured to facilitate boney in-growth.

In yet another aspect, a method for replacing at least a portion of a natural labrum about an acetabulum can include identifying a defect area in the natural labrum and removing at least a portion of the natural labrum that includes at least the defect area. A labrum implant can be provided to replace the at least a portion of the removed natural labrum. The labrum implant can include a body formed of a flexible polymer, which can include a base configured to engage a rim of the acetabulum, an upper side opposing the base, and an interior side extending from the base to the upper side and configured to conform to an arcuate shape of an articulating surface of a femoral head. The method can further include securing the body to at least the acetabular rim by passing an attachment member through at least one of a plurality of through bores formed in the body and extending through the base.

Further areas of applicability will become apparent from the description provided herein. It should be understood that the description and specific examples are intended for purposes of illustration only and are not intended to limit the scope of the present teachings.

### DRAWINGS

The present teachings will become more fully understood from the detailed description, the appended claims and the following drawings. The drawings are for illustrative purposes only and are not intended to limit the scope of the present disclosure.

FIG. 1 is a perspective view of a hip joint according to the present teachings;

FIG. 2 is a perspective view of a hip joint with an excessive growth or build-up on the femoral neck and femoral head according to the present teachings;

FIG. 3 is a perspective view illustrating a femoro-acetabular impingement condition according to the present teachings;

FIG. 4 is a perspective view illustrating a labral tear in a rim of the acetabulum according to the present teachings;

FIG. 5 is a perspective view of an exemplary labrum implant according to the present teachings;

FIG. 6 is sectional view of the exemplary implant of FIG. 5 according to the present teachings;

FIG. 7 is a perspective view of a procedure according to the present teachings;

FIG. 8 is a top plan view of an exemplary alternative configuration of the labrum implant according to the present teachings;

FIGS. 9 and 10 are sectional views of the labrum implant in conjunction with various exemplary alternative attachment configurations according to the present teachings;

FIGS. 11-11C illustrate various views of the labrum implant being secured with various alternative suture attachment configurations according to the present teachings;

FIG. 12 is a sectional view of an exemplary configuration of the labrum implant having a support member according to the present teachings;

FIG. 13 is a perspective view illustrating a labral tear in a rim of the acetabulum according to the present teachings;

FIG. 14 is a perspective view of an exemplary alternative labrum implant according to the present teachings;

FIG. 15 is a perspective view of an exemplary procedure according to the present teachings;

FIG. 16 is a perspective view of exemplary attachment configurations for the labrum implant according to the present teachings;

FIG. 17 is a sectional view of the labrum implant of FIG. 14 according to the present teachings;

FIG. 18 is a partial sectional view of the labrum implant of FIG. 14 being secured with a suture and suture anchor according to the present teachings; and

FIG. 19 is a perspective view of exemplary alternative attachment configurations for the labrum implant according to the present teachings.

### DETAILED DESCRIPTION

The following description is merely exemplary in nature and is not intended to limit the present disclosure, its application, or uses. It should be understood that throughout the drawings, corresponding reference numerals indicate like or corresponding parts and features, with the various elements within each view being drawn to scale.

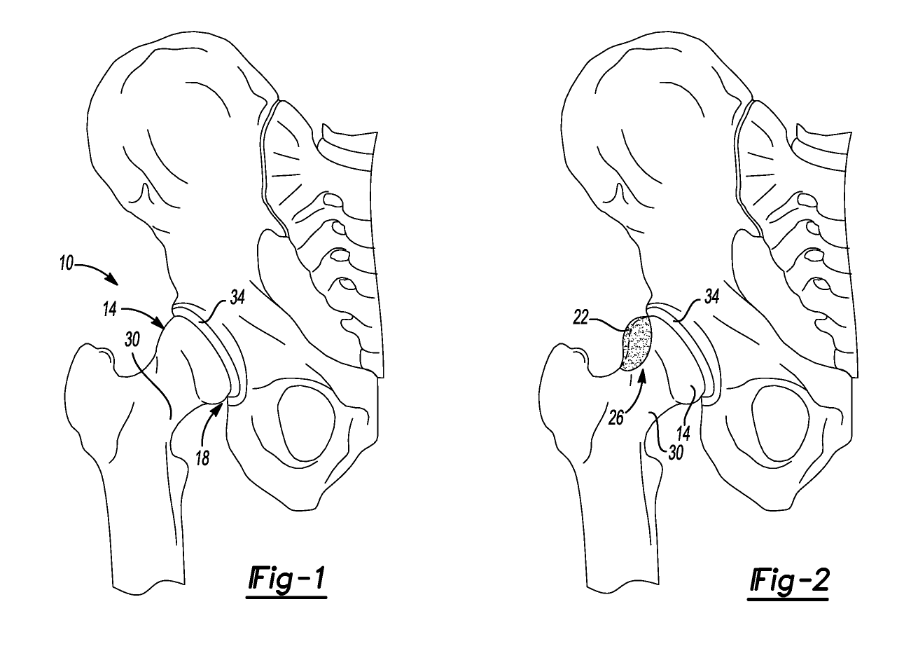

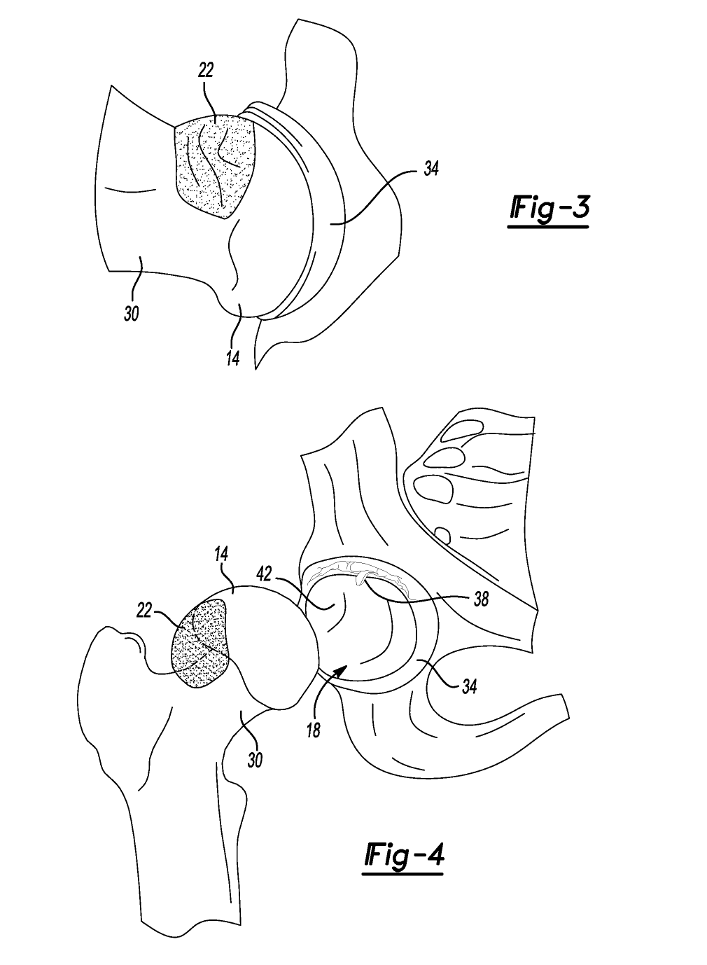

With initial reference to FIGS. 1-4, an exemplary hip joint 10 is illustrated where the femoral head 14 is seated in the acetabulum 18 for articulation thereabout, as shown in FIG. 1. In certain circumstances, an abnormal or excessive boney growth area or build-up 22 can occur in a region 26 along the femoral head 14 and femoral neck 30, as generally shown in FIG. 2. The boney growth area 22 can result in an impingement condition generally known as femoro-acetabular impingement (FAI) where the boney growth area 22 impinges on the acetabular rim surrounding the acetabulum 18 when the femoral head 14 articulates relative thereto, as generally shown in FIGS. 3 and 4. Such impingement can result in damage to the labrum 34, such as a labral tear 38 or the like, as well as damage over time to articular cartilage 42 of the acetabulum 18. Femoro-acetabular impingement can, if left untreated, eventually lead to a need to replace the hip joint with a prosthetic joint. As a result, it can be desirable to remove the boney growth area 22 by way of burrs, milling or osteotomes and repair or replace the damaged labrum.

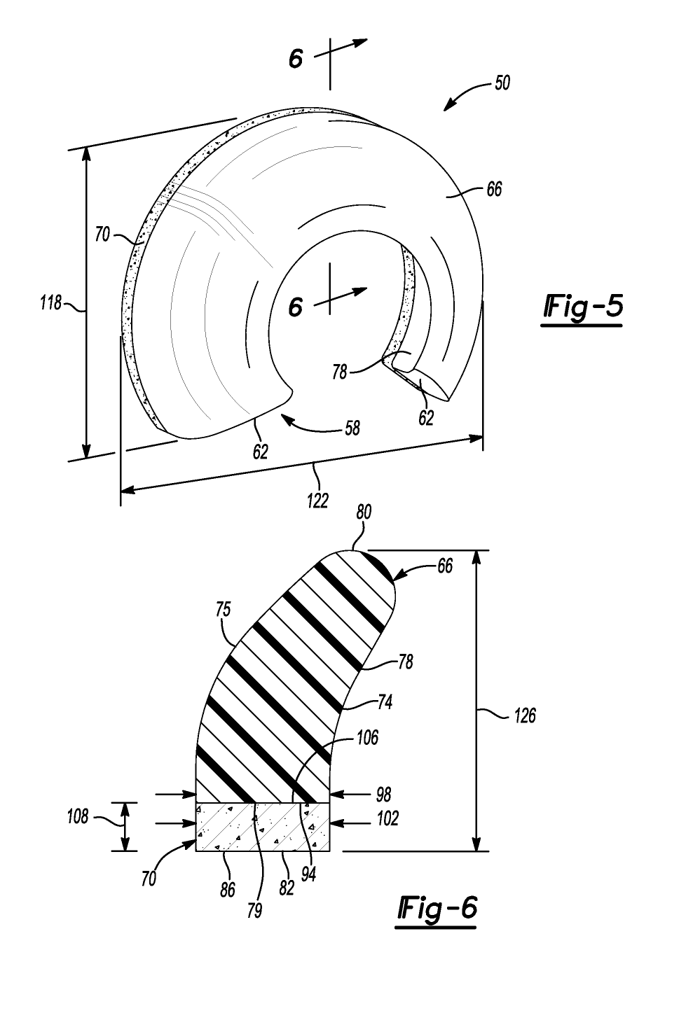

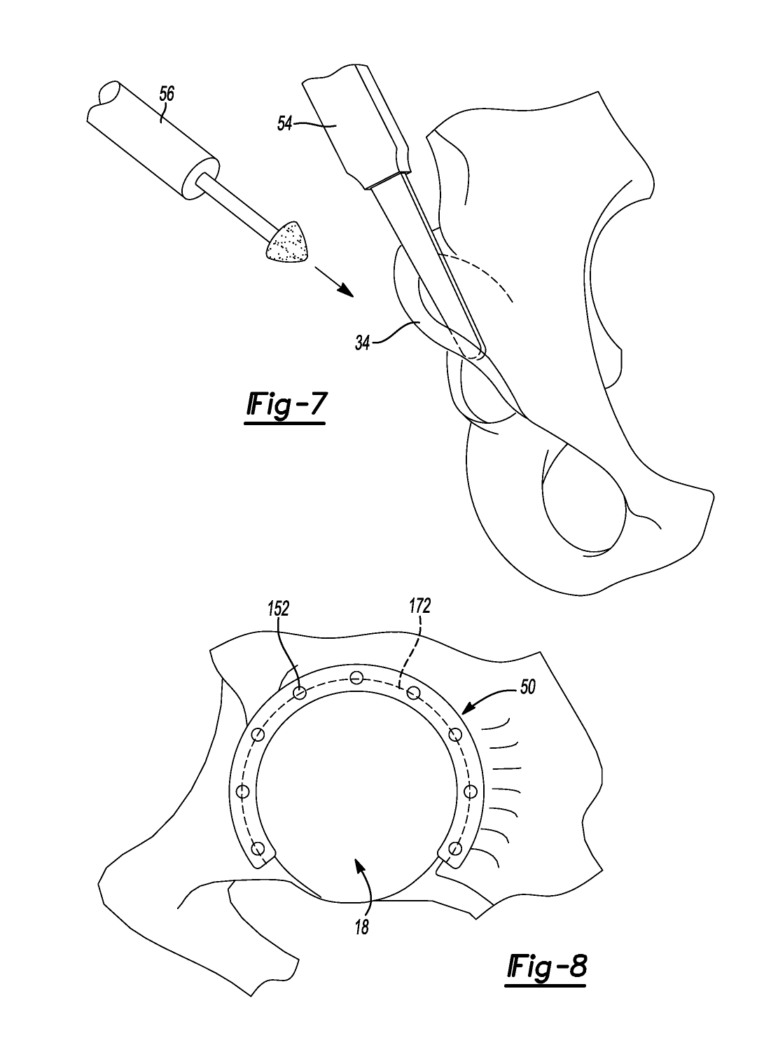

With additional reference to FIGS. 5-12, an exemplary labrum replacement implant 50 is shown for use in replacing the damaged labrum 34. When damage to the natural labrum 34 is extensive, as generally shown in FIG. 4, the implant 50 can be used to replace the entire labrum 34 after removing the damaged natural labrum 34 with an appropriate instrument, such as with a scalpel 54 generally shown in FIG. 7. It should be appreciated that while implant 50 is generally described in connection with replacing the entire natural labrum 34, implant 50 could be used for replacing only a portion of the natural labrum 34, as will be discussed herein.

The labrum implant 50 can include a generally C-shaped or horseshoe shaped configuration 58 with terminal ends 62, as generally shown in FIGS. 5 and 8. The implant 50 can extend from approximately 10-300 degrees to mimic the natural labrum, as generally shown in FIG. 5. The implant 50 can include a flexible sealing portion 66 and a support or base portion 70, as generally shown in FIG. 6. The flexible portion 66 can be formed of a flexible, biocompatible material such as silicone, polyurethane, polycarbonate urethane, a collagen matrix or the like. The flexible portion 66 can include an articulation side 74 configured to mimic the natural labrum and engage the articular cartilage of the femoral head 14, and an opposed side 75 having a generally convex shape. The articulation side 74 can include a concave arcuate shape or contoured articulation surface 78 generally complementary to the contour of the mating femoral head 14. The articulation surface 78 can sealingly engage the femoral head 14 to retain lubricating fluid in the acetabular socket for lubrication of the articulating femoral head 14. Flexible portion 66 can include a base side 79 and an opposed upper surface or end 80. Upper end 80 can be formed in various configurations including an arcuate surface, a pointed surface, a generally planar surface, etc.

The base 70 can include a generally rectangular shape 82 in cross-section having a bone engaging side 86 configured to engage the acetabular rim 90 and an opposite side 94 coupled to the base side 79 of flexible portion 66. Base 70 can further include a width 98 substantially equal to a width 102 of a base side 106 of flexible portion 66, and a thickness 108 suitable to provide appropriate backing, rigidity or support to the flexible portion 66 during articulation of the femoral head 14 relative thereto. It should be appreciated that while the base 70 has been described as having a generally rectangular shape 82 and a width 98 substantially equal to the width 102 of the of the flexible portion 66, base 70 can be provided in a variety of different shapes and sizes as may be required for implantation in labrum replacement procedures of various different patients.

In this regard, implant 50 can also be provided in various sizes generally directed towards the various sizes and configurations of potential patient recipients. For example, and with reference to FIGS. 5 and 6, implant 50 can be provided in a variety of different overall lengths 118 and widths 122, as well as widths 98 and 102 of base 70 and flexible portion 66, respectively. In addition, implant 50 can be provided with varying overall heights 126 in an effort to closely replicate the size of the natural labrum 34 to be replaced.

Base 70 can be formed of any biocompatible material of at least a semi-rigid nature to provide the above-mentioned support to flexible portion 66. The bone engaging side 86 of base 70, in a semi-rigid configuration, can therefore conform or accommodate surface irregularities in the acetabular rim 90. In one exemplary configuration, base 70 can be formed of a porous metal to enhance biologic fixation and bone in-growth, such as Regenerex® porous titanium construct 134, available from Biomet, Inc. of Warsaw, Ind. Alternatively, base 70 can be formed with a rigid or semi-rigid substrate and coated with a layer of Regenerex, or formed with a roughened surface, such as a plasma sprayed surface coating. In another exemplary configuration, base 70 can be formed of a resorbable polymer composition, such as LactoSorb®, also available from Biomet, Inc. of Warsaw, Ind. Base 70 can be fixed to the flexible portion 66 in any suitable manner, including using an adhesive or molding flexible portion 66 thereto.

In a configuration where base 70 includes a layer or coating of Regenerex or a roughened plasma sprayed surface, a side 142 of the base 70 that would be facing the femoral head upon implantation can remain free of such coating or roughened surface so as to not come into contact with the articular cartilage of femoral head 14. Alternatively, side 142 can be recessed from the articulation surface 78 such that side 142 would not contact the femoral head 14 upon implantation. Such a configuration can be used with the layers and coating discussed above, as well as in the configuration where base 70 is formed out of a porous construct, such as Regenerex.

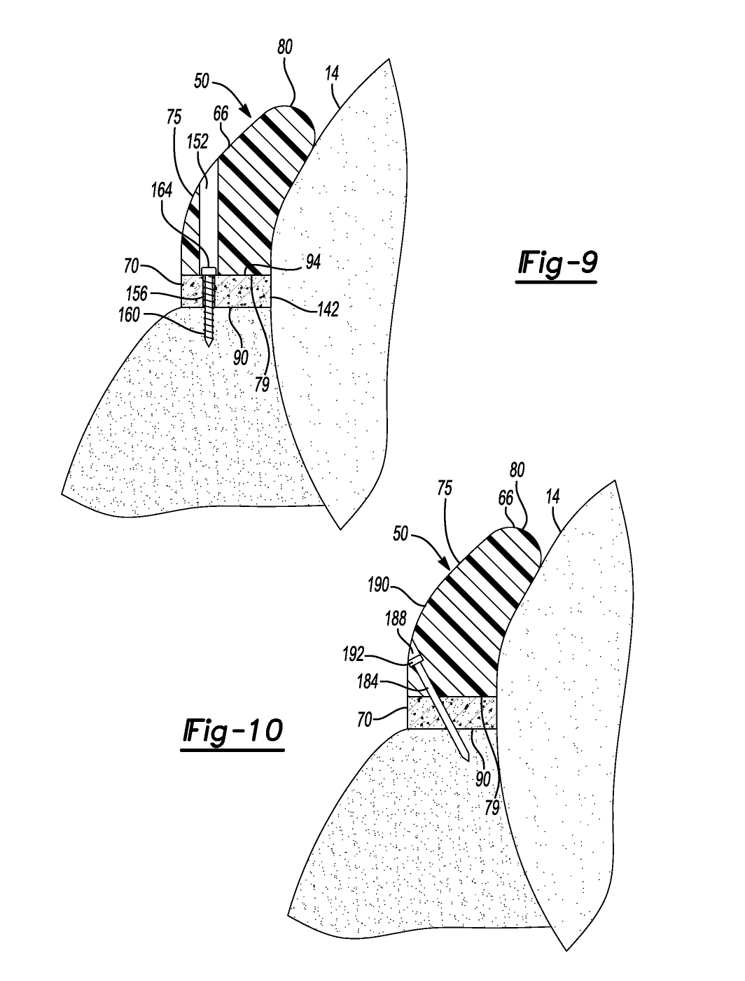

With additional reference to FIGS. 8-11, various features for use in fixing implant 50 to the acetabular rim 90 will now be described. With particular reference to FIG. 9, implant 50 can include and define a plurality of bores 152 formed in flexible portion 66 and aligned with a corresponding bore 156 in base portion 70. Bores 152 and 156 can be sized to receive a bone fastener, such as a bone screw 160, for use in affixing labrum implant 50 to the acetabular rim 90. In one exemplary configuration, bores 152 can include a first diameter larger than a second diameter of bores 156 such that a head 164 of fastener 160 will abut side 94 of base 70 and fix implant 50 to the acetabular rim 90 upon engaging fastener 160 therewith. Bores 152 and 156 can be positioned along a central region 172 of implant 50, as generally shown in FIG. 8, or can be positioned in an alternating configuration about central region 172 where the bores are positioned closer to edges 176 and 180 of implant 50.

In an alternative configuration, implant 50 can be fixed to the acetabular rim 90 using a surgical tack or tacks 184 or the like. Tacks 184 can include a length sufficient to be driven through a portion of the flexible portion 66 and base 70, as generally shown in FIG. 10. Tacks 184 can be driven though the flexible portion 66 and base 70 of implant 50 without requiring a preformed bore, such as bores 152 and 156. Implant 50 can include a plurality of recesses or depressions 188 in an outer perimeter 190 thereof to facilitate receipt of a head portion 192 of tacks 184. Tacks 184 can be used alone or in combination with fasteners 160 to fix implant 50 to the anatomy. In one exemplary configuration, implant 50 can be secured to the acetabular rim using bone fasteners 160 along central region 172 and the tack or tacks 184 along the outer perimeter 190, as shown in FIG. 10. It should also be appreciated that tacks 184 can be received in bores 152 and 156 to secure implant 50 to acetabular rim 90.

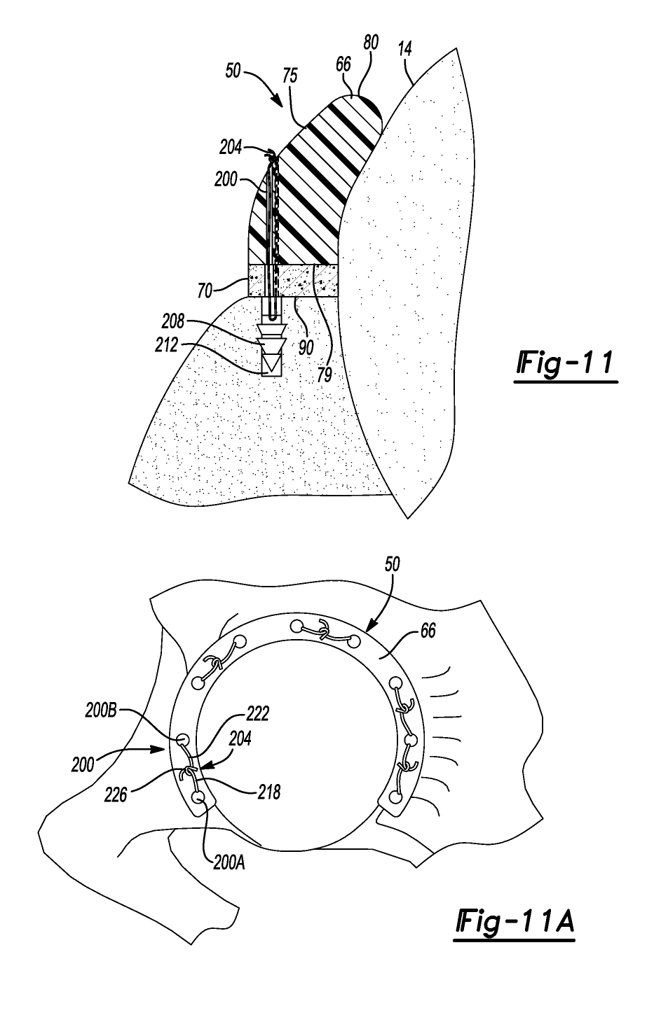

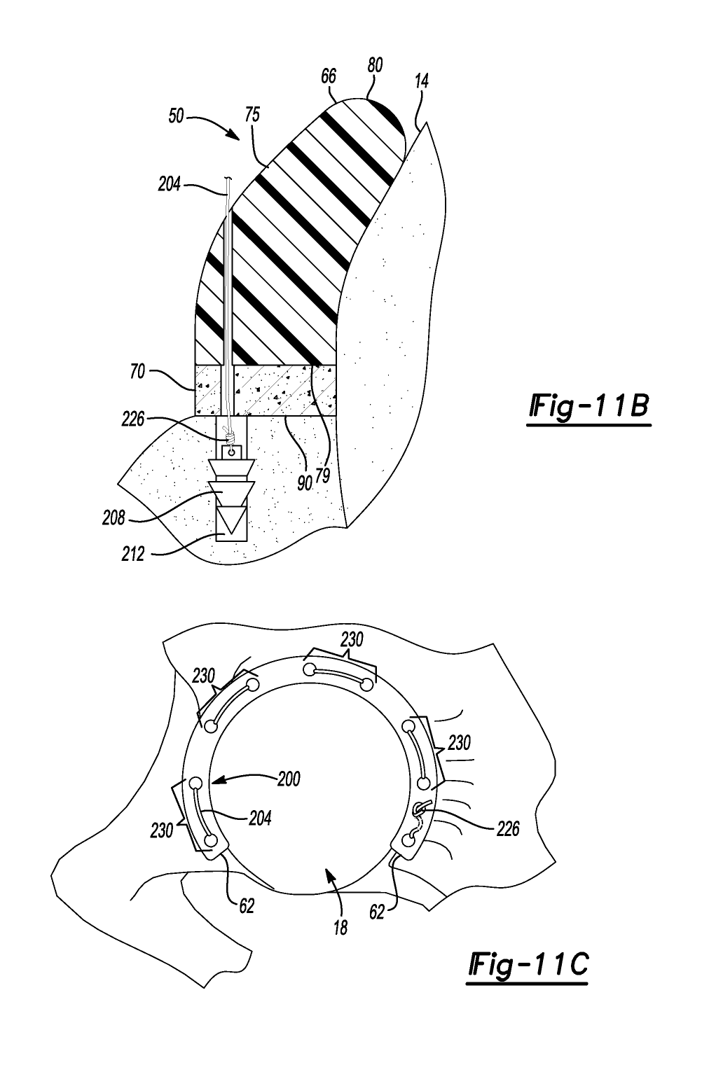

With particular reference to FIG. 11, implant 50 can include a plurality of bores 200 configured to receive a flexible construct or suture 204 therethrough for securing implant 50 to the acetabular rim 90. Suture bores 200 can extend through the flexible portion 66 and base 70 and can be placed around the implant, as generally shown in FIG. 11A. A plurality of suture anchors, such as exemplary suture anchor 208, can be secured to prepared bores 212 in the acetabular rim 90 and used to secure suture 204 and implant 50 to the bone. In one exemplary configuration, a single suture 204 can be used to secure a portion of implant 50 to the bone using a single suture anchor 208. In this configuration, suture 204 can be passed through anchor 208 with one free end 218 extending though one bore 200A and another free end 222 extending though another bore 200B such that the ends 218, 222 can be tied with a knot 226 or the like to secure implant 50 to acetabular rim 90, as generally shown in FIGS. 11 and 11A. In this manner, a plurality of sutures 204 and corresponding suture anchors can be used along a length of implant 50 to secure the entire implant 50 to the acetabular rim 90.

In an alternative configuration, a single suture 204 can be used to secure implant 50 to a plurality of suture anchors 208, where the plurality of suture anchors 208 can be placed around the acetabular rim in prepared bores 212 such that, for example, one suture anchor 208 can be provided for every pair of suture bores 230 in the implant 50. The single suture can be secured to a first anchor proximate one terminal end 62 of implant 50 and can be threaded through additional anchors 208 and associated pairs of suture bores 230, as generally shown in FIGS. 11B and 11C. The single suture 204 can be secured with a knot or the like to the other terminal end 62, as also shown in FIG. 11C. It should be appreciated that more or less suture anchors 208 can be used as determined by a surgeon during the implantation procedure. In this regard, it should also be appreciated that while the implant 50 is shown having a plurality of bores 200, such a plurality of bores can be provided to offer flexibility to the surgeon for both location and number of suturing points that can be used during a procedure and thus it may not be necessary to use all of the bores 200.

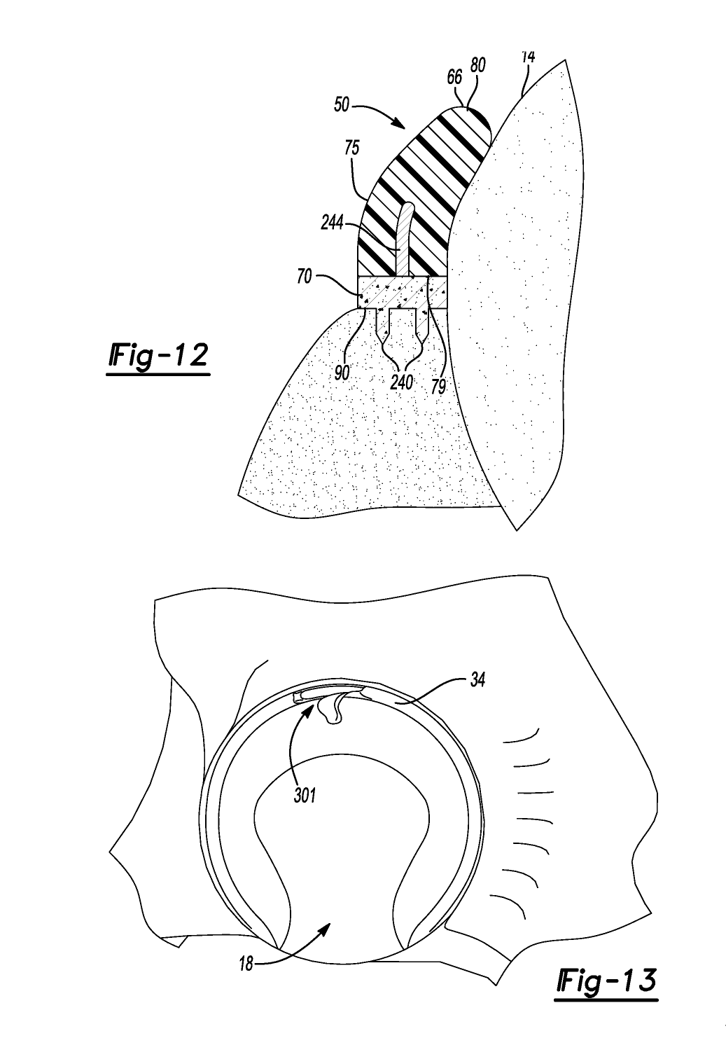

With additional reference to FIG. 12, implant 50 is shown with a pair of projections or spikes 240 extending from the bone engaging side 86 of base 70 for use in facilitating engagement and retention of implant 50 to acetabular rim 90. Projections 240 can be integrally formed with base 70 or can be a separate member affixed thereto when the base 70 is formed. Projections 240 can include the coating of Regenerex or a roughened surface as described above and can be used in combination with any of the above-discussed attachment features. It should be appreciated that while two projections are shown positioned laterally across the width 98 of base 70, various numbers and placement configurations of the projections 240 can be provided to accommodate various different anatomical configurations. For example, projections 240 can be located about a periphery of base 70 or could be positioned at ends of base 70 adjacent the terminal ends 62 of implant 50.

Implant 50 can also include an optional support member or rib 244 that extends internally along a length of the implant, as generally shown in the cross-sectional view of FIG. 12. Support rib 244 can extend substantially along the length of implant 50 from one terminal end 62 to the other terminal end 62, or along a portion of the length therebetween. Support rib 244 can also include one rib that extends as discussed above, or multiple ribs 244 extending between the terminal ends 62. Support rib 244 can aid in maintaining the shape of the flexible portion 66 over time. The support rib 244 can be molded into the flexible portion 66 and/or can be an integral portion of base 70.

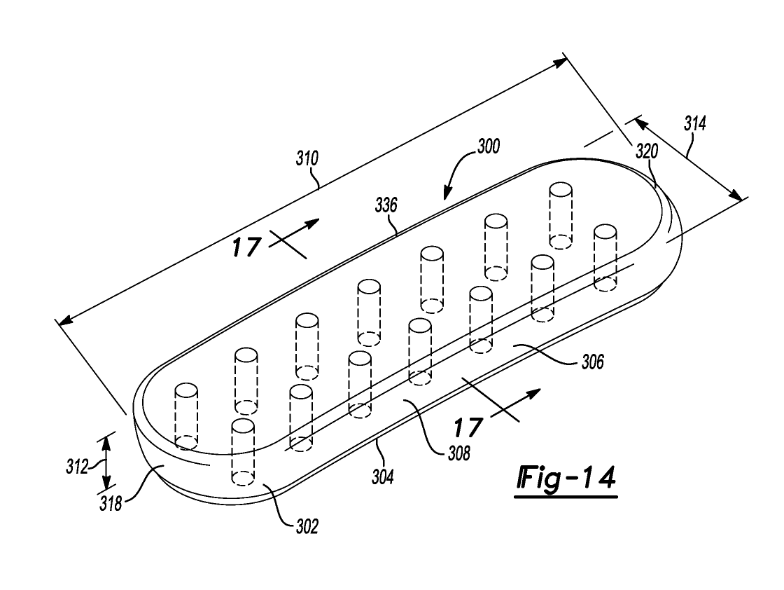

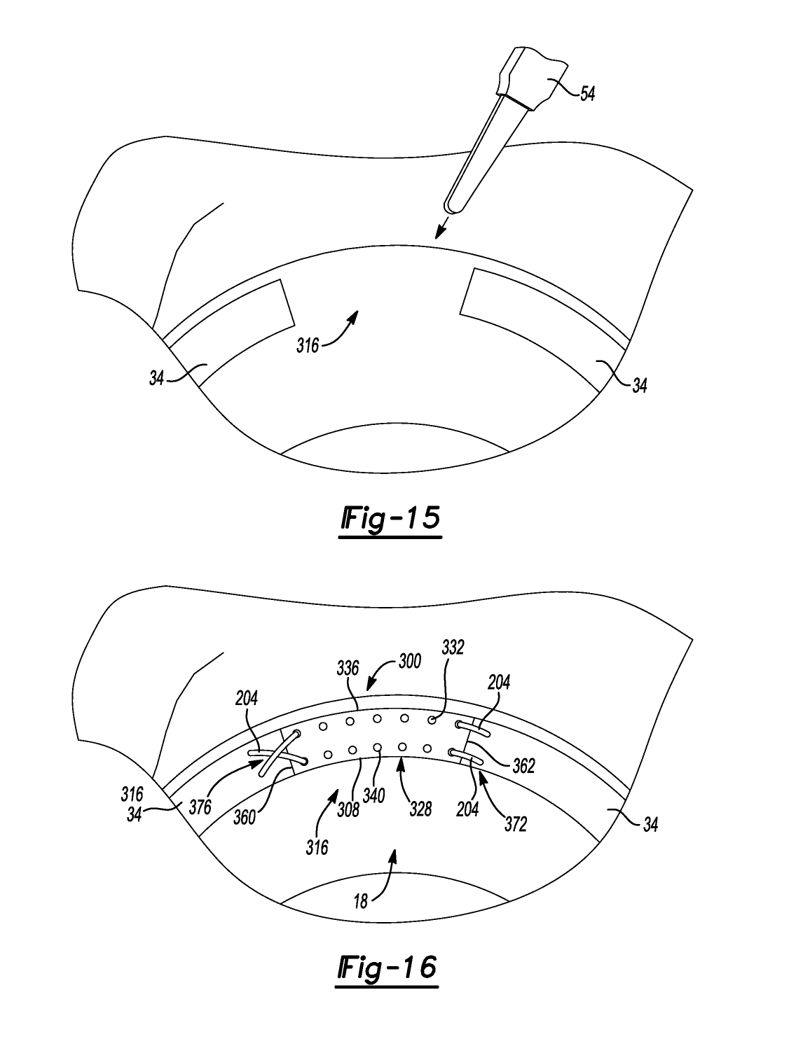

With additional reference to FIGS. 13-19, an alternative implant 300 for repairing a damaged portion of the natural labrum 34 will now be described. Implant 300 can also be formed of various biocompatible materials that are flexible and pliable, such as silicone, to mimic the natural labrum. In one exemplary configuration, implant 300 can be used for replacing only a damaged area or portion 301 of the natural labrum 34, as generally shown in FIGS. 13 and 15. In this regard, implant 300 can be provided in various sizes and configurations to be selected and shapeable, as can be required, by a surgeon to conform to the surrounding natural labrum, as will be described in greater detail below. For example, implant 300 can include a generally rectangular configuration 302 with a generally planar bone-engaging surface 304 and a slight arcuate shape 306 on an interior side 308 that is configured to be in articulating engagement with the femoral head 14, as generally shown in FIG. 14. Implant 300 can be provided in various configurations including different combinations of lengths 310, thicknesses 312 and widths 314. From these various configurations, a surgeon can select a size of implant 300 that most closely corresponds to the damaged labral portion 301 and/or a prepared implant site 316 (FIG. 15), as will be discussed in greater detail below.

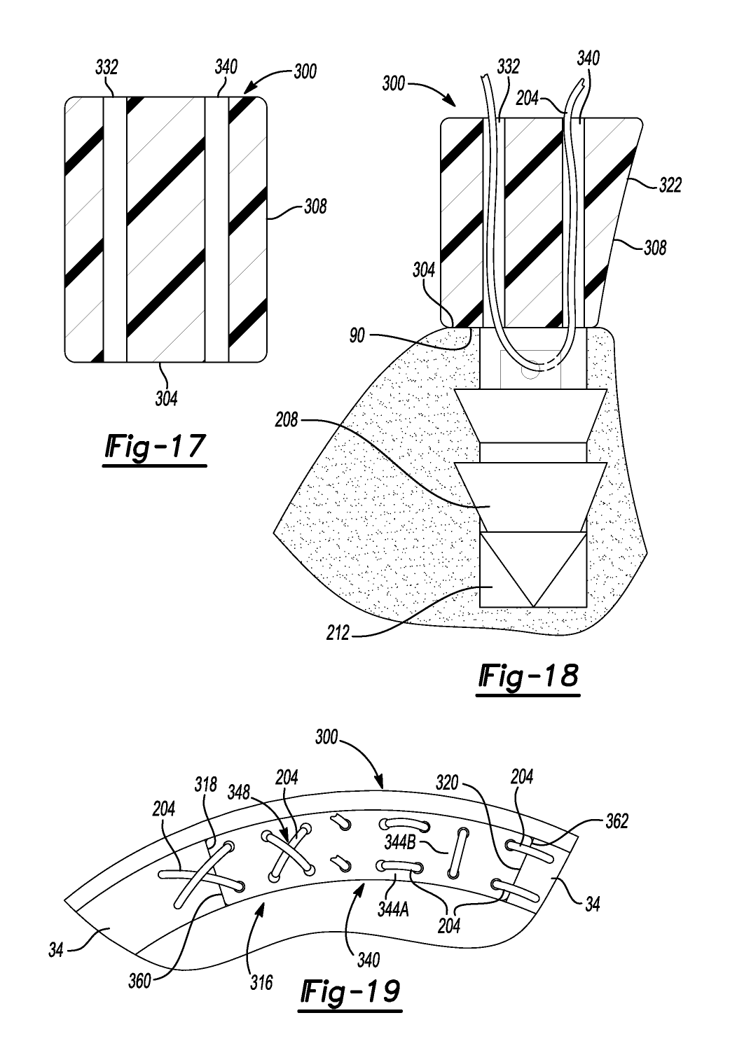

Depending on the nature of the damaged area (i.e., proportion of damaged labrum to surrounding non-damaged labrum) as well as various other factors, a surgeon can choose to prepare the implant site 316 to closely match the nearest selectable size of implant 300 that corresponds to a size of only the damaged portion 301. Alternatively, if it is determined that implant site 316 requires specific dimensions that do not exactly correspond to a selectable size of implant 300, the implant can be sized to match implant site 316. For example, and with reference to FIGS. 14-16, first and second ends 318, 320 can be trimmed, as needed to match implant site 316. In addition, interior side 308 can be trimmed/shaped to substantially conform to the femoral head 14. In this manner, interior side 308 can be shaped to have an inclined or arcuate shape 322 that closely corresponds to a patient's femoral head 14, as generally shown in FIG. 18.

It should also be appreciated that the flexible and pliable nature of implant 300 provides for the bone engaging surface 304 to be able to substantially conform to the acetabular rim 90. In this manner, implant 300 can accommodate surface irregularities of a moderate nature such that acetabular rim 90 does not necessarily need to be prepared to have a planer surface for receipt of implant 300, as will be discussed below in greater detail. Further, the flexible and pliable nature of implant 300 allows for the implant to be positioned in a generally arcuate configuration along the acetabular rim 90, as generally shown in FIGS. 16 and 19.

Implant 300 can include a plurality of bores 328 configured to receive a suture or other securing devices therein to secure implant 300 to at least the acetabular rim 90. In an exemplary configuration, implant 300 can include a first row or set of bores 332 positioned adjacent an exterior side 336 of implant 300 and a second set of bores 340 positioned adjacent interior side 308, as shown in FIGS. 14 and 16. By placing the first and second set of bores adjacent respective exterior and interior sides 336, 306, the implant 300 can be secured to the acetabular rim 90 near its respective sides to substantially prevent lifting of the implant 300 at its edges.

The plurality of bores 328 in the exemplary first and second sets 332, 340, can provide various attachment location and configuration options to a surgeon. In an exemplary configuration, implant 300 can be secured to the acetabular rim 90 with suture 204 and suture anchor 208, as generally shown with reference to FIGS. 18 and 19. As discussed above with reference to implant 50, while only one suture anchor 208 is shown in FIG. 18, a plurality of suture anchors 208 can be provided to secure implant 300, where the number of anchors being used can depend on, for example, the size of the implant 300 and/or the surgeon's preference. In an exemplary configuration, one suture anchor 208 can be provided for two laterally opposed suture bores 328, as generally shown in FIG. 18. In this configuration, suture 204 can be passed through one bore from the first set 332 and another bore from the second set 340. It should be appreciated however that more than two bores 328 can be associated with a suture anchor 208.

In addition to using two laterally opposed bores, various other configurations can be utilized to secure a portion or all of implant 300 to the acetabular rim 90, as generally shown with reference to FIG. 19. Different suture securing configurations may be required depending on various factors including limitations on where suture anchors can be positioned in the acetabular rim 90. For example, a suture securing configuration 340 can be utilized where suture 204 is fed through the holes in an alternating configuration traveling around the perimeter of implant 300. In this manner, suture 204 can be passed over a portion 344A of implant 300 between adjacent bores 328 of set 332, then down to a suture anchor 208, then back over another portion 344B, then back down to the same or another suture anchor 208, etc. continuing around the entire perimeter of implant 300. In another exemplary configuration, a cross stitch pattern 348 can be utilized, as also shown in FIG. 19. It should be appreciated that the various combinations of different suture securing configurations or patterns described above, including the perimeter configuration 340, the cross stitch pattern 348, and the lateral bore configuration can be used independently or in various combinations with each other.

In addition to securing implant 300 to the acetabular rim 90 as discussed above, implant 300 can also be secured to mating sides 360, 362 of the natural labrum 34, as generally shown in FIGS. 16 and 19. Various suture securing configurations can also be used to secure implant 300 to the surrounding labrum 34, including a parallel configuration 372 and/or a cross-over configuration 376. It should be appreciated that the bores 328 used for securing implant 300 to adjacent sides 360, 362 of labrum 34 can also be used in combination therewith to secure implant 300 to the acetabular rim 90, as discussed above. While implants 50 and 300 have been described above in conjunction with various attachment options and features, it should also be appreciated that the various attachment features and options can be mixed and matched for each implant 50 and 300, as well as between the implants 50 and 300.

With general reference to FIGS. 1-19, a procedure associated with implants 50 and/or 300 will now be described. As generally discussed above, FAI can cause damage to labrum 34 that can necessitate replacement of the entire labrum 34 (FIGS. 4 and 7) or only a portion of the labrum 34 (FIGS. 13 and 15). In either scenario, the entire labrum or respective portion can be removed with an appropriate instrument, such as scalpel 54 shown in FIGS. 7 and 15

Once the natural labrum 34 or a portion thereof has been removed, the underlying surface of the acetabular rim 90 can be prepared for receipt of implant 50 or 300. It should be understood that while implant 50 has been generally described as replacing the entire labrum 34 and implant 300 has been generally described as replacing a portion of the labrum 34, either implant could be used to replace a portion or the entire labrum, as discussed herein. For discussion purposes only, however, the procedure will be described with reference to implant 50 replacing the entire labrum and implant 300 replacing a portion of the labrum, with the understanding that either implant can be used to replace a portion or all of natural labrum 34.

For implant 50, the acetabular rim 90 can be prepared with an appropriate instrument, such as a burr or mill 56, to provide a substantially planar surface for receipt of the substantially planar bone engaging side 86, as generally shown with reference to FIGS. 6-7 and 9. One of the various sizes of implant 50 that most closely corresponds to the removed natural labrum 34 can be selected for implantation. It should be appreciated that the base 70 of implant 50 can be formed to be partially compliant such that the width 122 of a selected implant 50 could be slightly adjusted, if required, to align with a patient's acetabular rim 90.

Once a specific implant 50 has been selected, the selected implant can be secured to the acetabular rim 90 using one of the above attachment options. In this regard, the implant 50 can be secured using bone fasteners 60 (FIG. 9), tacks 184 (FIG. 10), suture 204 with corresponding anchors 208 (FIGS. 11-11C), projections 240 (FIG. 12), or combinations thereof. If implant 50 is to be secured with at least one suture anchor 208, appropriately positioned bores 212 can be prepared in the acetabular rim 90, such as with a reamer, for receipt of anchor 208 in an interference fit configuration, as generally shown for example in FIG. 11.

In a similar manner, implant 300 can be selected to replace the damaged portion 301 of labrum 34 shown in FIG. 13 where the damaged portion 301 can be removed, as discussed above and shown in FIG. 15 with reference to implant site 316. As also discussed above, implant site 316 can be sized to substantially correspond to an available size of implant 300, or implant 300 can be sized to correspond to a specific size of implant site 316, or combinations of both.

Once implant site 316 has been formed, the exposed portion of acetabular rim 90 can be prepared for receipt of the selected implant 300. As also discussed above, the flexible and pliable nature of implant 300 can adapt to surface irregularities in acetabular rim 90 such that minimal, if any forming of the bone may be required for receipt of implant 300. In this regard, a surgeon can evaluate the implant site 316 and determine if any milling or other surface forming procedures are required for the exposed acetabular rim 90. The selected implant 300 can then be secured in implant site 316 with one or a combination of the various suture attachment options discussed above.

As discussed above, the surgeon can determine the number of suture anchors 208 to be used as well as appropriate locations for the anchors in the acetabular rim 90. Corresponding suture anchor bores 212 can then be prepared for receipt of the corresponding suture anchors 208. Implant 300 can then be secured to the acetabular rim 90 using one of the various suture securing configurations discussed above. Implant 300 can also be secured to respective mating sides 360, 362 with any suitable suture configuration, including the parallel and/or cross-over suture securing configurations 372, 376 discussed above. It should be appreciated that implant 300 can be secured only to the acetabular rim 90 or to the acetabular rim 90 and natural labrum 34. In addition, implant 300 can be secured to acetabular rim 90 before or after being secured to the mating sides 360, 362 of the labrum 34. Further, implant 90 can be secured to acetabular rim 90 directly with suture 204 without using suture anchors 208.

While one or more specific examples have been described and illustrated, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof.

What is claimed is:

1. A labrum implant configured to replace at least a portion of a natural labrum about an acetabulum, comprising:
a body portion formed of at least a biocompatible flexible polymer, the body portion including a base, an opposed upper end opposite the base, an interior femoral head engaging side, and an opposed exterior side, the interior side having an arcuate shape in cross-section adapted to conform to an articular surface of a femoral head, the body portion having an arcuate shape in plan view adapted to conform to at least a portion of the rim of the acetabulum and defining a plurality of bores extending therethrough from the upper surface or the exterior side to the base; and
an attachment member having at least a portion coupled to at least one of the bores and adapted to secure the implant to the acetabular rim such that the base engages the acetabular rim.

2. The implant of claim 1, further comprising a support portion formed of at least a semi-rigid biocompatible material and having a first side adapted to engage the acetabular rim and an opposing second side fixed to the body portion, the support portion including a plurality of bores extending therethrough and aligned with the plurality of bores of the body portion;
wherein at least the first side includes a roughened or porous surface configured to facilitate boney in-growth.

3. The implant of claim 2, wherein the support portion includes a width in cross-section substantially equal to a width of the base of the body portion.

4. The implant of claim 3, wherein the interior side of the body portion includes a first end adjacent the base and a second opposite end adjacent the upper side, the second end extending laterally beyond the width of the base and the support portion in cross-section.

5. The implant of claim 2, wherein the support portion and the body portion together form the implant in a unitary configuration, the implant having a generally C-shape or horseshoe shape in plan view; and
wherein the body portion is formed from polyethylene, polycarbonate urethane or silicone, and the support portion is formed of porous titanium.

6. The implant of claim 2, wherein at least one of the plurality of bores of the body portion include a first diameter larger than a second diameter of the corresponding plurality of bores in the support portion; and
wherein the attachment member includes a bone fastener having a shaft and a head, the head having a diameter larger than the second diameter, and the fastener configured to be received in at least one of the bores of the body portion such that the shaft passes through a corresponding bore in the support portion and the head engages the second side of the support portion to secure the implant to the acetabular rim.

7. The implant of claim 2, wherein the attachment member includes at least one suture coupled to a suture anchor that is adapted to be secured to the acetabular rim, the at least one suture configured to be passed through at least two of the plurality of holes to secure the implant to the acetabular rim.

8. The implant of claim 2, further comprising a support member extending from the second side of the support portion and into the body portion so as to be surrounded by the body portion.

9. The implant of claim 2, further comprising at least one projection extending from the first side of the support portion and adapted to engage the rim of the acetabulum.

10. The implant of claim 1, wherein the plurality of bores includes a first row of spaced apart bores adjacent the exterior side and a second row of spaced apart bores adjacent the interior side of the body portion.

11. The implant of claim 10, wherein the attachment member includes at least one of a suture, a bone tack, a bone fastener, or combinations thereof configured to be received in at least one of the plurality of bores, the suture configured to be received in at least two of the plurality of bores of at least one of the first and second rows and engage a suture anchor to secure the implant to the acetabular rim.

12. A labrum implant configured to replace at least a portion of a natural labrum about an acetabulum, comprising:
a body portion formed of at least a biocompatible flexible polymer, the body portion including a base, an opposed upper end opposite the base, an interior femoral head engaging side, and an opposed exterior side, the interior side having a shape in cross-section adapted to conform to an articular surface of a femoral head, the body portion having an arcuate shape in plan view adapted to conform to a rim of an acetabulum; and
a support portion formed of at least a semi-rigid biocompatible material and having a first side adapted to engage the acetabular rim and an opposing second side fixed to the body portion, the support portion having an arcuate shape complimentary to the arcuate shape of the body portion;
wherein at least the first side of the support portion includes a roughened or porous surface configured to facilitate boney in-growth.

13. The implant of claim 12, further comprising a plurality of bores formed through the body portion and the support portion; and
an attachment member arranged to be coupled to at least one of the plurality of bores and adapted to engage the acetabular rim to secure the implant thereto.

14. The implant of claim 13, wherein the attachment member includes a bone tack, a bone screw, a suture, a suture anchor, or combinations thereof.

15. The implant of claim 12, wherein the body portion is formed of polyethylene, polycarbonate urethane or silicone, and the support portion is formed of porous titanium.

16. The implant of claim 12, further comprising a support member extending from the second side of the support portion and partially into the body portion so as to be surrounded by the body portion; and
at least one projection extending from the first side of the support portion and adapted to engage the rim of the acetabulum.

17. The implant of claim 12, wherein the support portion and the body portion together form the implant in a unitary configuration, the implant having a generally C-shape or horseshoe shape in plan view; and
wherein the interior side of the body portion includes a first end adjacent the base and a second opposite end adjacent the upper side, the second end extending laterally beyond a width of the base and the support portion in cross-section.

18. The labrum implant of claim 12, wherein the interior side of the body portion includes a concave shape in cross-section and the exterior side includes a convex shape in cross-section.

19. A method for replacing at least a portion of a natural labrum about an acetabulum, comprising:
identifying a defect area in the natural labrum;
removing at least a portion of the natural labrum that includes at least the defect area;
providing a labrum implant to replace the at least a portion of the removed natural labrum, the labrum implant having a body formed of a flexible polymer and including a base configured to engage a rim of the acetabulum, an upper side opposing the base, and an interior side extending from the base to the upper side and configured to conform to an arcuate shape of an articulating surface of a femoral head; and
securing the body to at least the acetabular rim by passing an attachment member through at least one of a plurality of through bores formed in the body and extending through the base.

20. The method of claim 19, wherein securing the implant to at least the acetabular rim includes forming a bore in the acetabular rim configured to receive a suture anchor and passing a suture through at least one of the body bores and coupling the suture to the suture anchor secured in the acetabular rim to secure the implant thereto.

21. The method of claim 20, further comprising securing opposed ends of the implant body to mating adjacent portions of the natural labrum with a suture.

22. The method of claim 19, further comprising shaping at least the interior side of the body to substantially conform to the articulating surface of the femoral head.

23. The method of claim 19, wherein providing a replacement implant includes providing a replacement implant formed from polycarbonate urethane or silicone.

24. The method of claim 19, wherein providing a labrum replacement implant further includes providing a support portion having a first side integrally coupled to the base of the body and a second opposing side configured to engage the acetabular rim, the support portion being formed to include at least an exterior coating of porous metal and a plurality of through bores configured to align with the plurality of bores in the body.

25. The method of claim 24, wherein removing at least a portion of the natural labrum includes:
removing the entire natural labrum; and
preparing the underlying acetabular rim to have a substantially planar surface for receipt of the second side of the support portion.

26. The method of claim 24, further comprising providing the interior side of the body with an arcuate shape in cross section configured to substantially conform to the shape of mating femoral head.

27. The method of claim 24, wherein providing a labrum replacement implant includes providing the body integrally formed with the support portion such that the implant has a generally C-shape or horseshoe shape in plan view.

28. The method of claim 24, wherein securing the body to at least the acetabular rim by passing an attachment member through at least one of a plurality of through bores includes passing a bone fastener through one of the bores in the body portion such that a head of the fastener engages the support portion to secure the implant to the acetabular rim.

29. The method of claim 24, further comprising positioning the implant relative to the femoral head such that the interior side of the body engages the femoral head and performs a sealing function to substantially prevent passage of fluid between the implant and femoral head so as to retain lubricating fluid within the acetabulum.
